(19) United States
(12) Patent Application Publication
(10) Pub. No.: US 2010/0144625 A1
(43) Pub. Date: Jun. 10, 2010

(54) Organic Compounds

(76) Inventor: Rudi Mueller-Walz, Schopfheim (DE)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/515,130

(22) PCT Filed: Nov. 12, 2007
(86) PCT No.: PCT/EP2007/009761

§ 371 Date: Dec. 21, 2009

(30) Foreign Application Priority Data
Nov. 15, 2006 (GB) 0622818.3

(51) Int. Cl. A61K 38/16; A61K 38/02; A61K 31/7052; A61K 31/715; A61P 11/00

(52) U.S. Cl. 514/12; 514/2; 514/44 R; 514/54

(57) ABSTRACT

A powder formulation for inhalation comprising a macromolecule and a crystalline carrier material.

This invention is concerned with dry powder formulations for pulmonary delivery of biologically active macromolecules (herein after ‘macromolecules’), and to methods of producing same.

The delivery of macromolecules by inhalation attracts increasing amounts of attention due to the advantages the lung offers as a delivery route compared with conventional parenteral delivery.

However, there are certain technical hurdles that must be overcome before inhalation formulations of macromolecules can realize their potential as successful therapies in the market place: Bulk macromolecule-containing material must be reduced in size to form fine particles that are sufficiently small to be inhaled into the deep lung, where they can then be delivered systemically. However, fine particles generally have poor bulk properties and by themselves cannot be handled and filled into dispensing devices. Accordingly, in order to provide inhalable formulations, the fine particles have to be blended with carrier materials that help to separate the fine particles, and also control the bulk properties of the inhalable formulation (such as flow properties and the like). Blending fine particles with coarse carrier materials is a complicated process if intimate mixtures are to be formed without affecting the integrity of the macromolecules. Blending processes can be protracted and require a large energy input into the blend, particularly if the bulk material is sticky or cohesive, as macromolecules often are.

Still further, inhalable formulations must be capable of being stored for long periods of time without adversely affecting the bulk properties of the formulation or the biological activity of the macromolecule, if the formulation is to be capable of being dispersed from a dry powder inhaler device with a high proportion of biologically active macromolecule in fine particle form with consistent and reliable dose uniformity.

The prior art contains examples of low molecular weight synthetic pharmaceutical agents that have been formulated as fine particles for inhalation. However, formulating macromolecules is more complex owing to the often extremely labile nature of macromolecules, and the often sticky or cohesive nature of macromolecules in bulk form.

The labile nature of macromolecules is partly a result of their activity being dependent on the way that they fold to form 3-dimensional structures. This is sometimes referred to as the “tertiary structure” of the macromolecule. The tertiary structure can be disrupted (often irreversibly) with very small inputs of energy. In summary, macromolecules are generally not very robust, and given the harsh conditions often needed for particle size reduction, and subsequent blending, the formulation of macromolecules in inhalable form presents a difficult challenge to formulators.

Accordingly, there is a need to provide methods of blending fine particles containing macromolecules with carrier materials, without destroying the biological activity of the macromolecules. Furthermore, there is a need for inhalation formulations in the form of dry powders that possess good bulk properties, which provide a stable medium for the macromolecule, and which are capable of dispensing the macromolecule with high fine particle fraction, consistently and reliably even after prolonged periods of storage.

There is also a need to provide methods of reducing the particle size of bulk macromolecule material in order to provide macromolecule-containing particles of sufficiently small mean mass diameter to be delivered into the deep lung, without disrupting the biological activity of the macromolecule.

Furthermore, having regard to the expense of many bulk macromolecules, the methods and formulations must be achieved in a simple and cost effective manner.

There is little teaching in the prior art relating to the formulation of fine particles containing macromolecules suitable for pulmonary delivery. U.S. Pat. No. 6,051,256 (herein after “256”). '256 describes powder formulations containing macromolecules, and a method of producing same. The powders are formed using spray drying techniques. Spray drying consists essentially of the steps of making a solution, slurry or suspension of the macromolecule, atomizing the solution, slurry or suspension to form particles and drying the particles.

Conventional spray drying exerts rigorous chemical, mechanical and thermal stresses on macromolecules, and so does not seem well suited for the formulation of labile macromolecules. In solution macromolecules are susceptible to chemical degradation and may need to be stabilized with, for example sugars, buffers, salts and other proteins. Further, shear forces during atomization and drying creates mechanical and thermal stresses on macromolecules. Nevertheless, by carefully controlling the spray-drying conditions, '256 claims that deficiencies of the conventional spray drying process can be remedied in order to render the technique more amenable for producing fine particles containing macromolecules. However, these deficiencies are only remedied by means of a complicated, multi-step process, at each stage of which it is necessary to carefully control process parameters and conditions. Accordingly, whereas 256 claims to form acceptable particles, the process described, and the in-process control is relatively complex, and not cost efficient. Given the high cost of macromolecules, the formulator does not have the latitude to perform complex formulation techniques if a cost effective formulation is to be realised.

Other particle-size reduction techniques include precipitation techniques, and micronisation techniques known in the art.

Precipitation techniques are complicated: The correct solvent conditions must be selected to give the required particle size. Furthermore, the formulator's latitude to adopt appropriate solutions to achieve desired particle size may be limited when formulating macromolecules, because of the chemically labile nature of the materials. Nevertheless, U.S. Pat. No. 6,051,256 and U.S. Pat. No. 5,981,719 report protein-containing microparticles. Similarly, Biosphere® microparticles as more fully described in U.S. Pat. No. 4,822,535; U.S. Pat. No. 6,120,787; WO02/28908; WO 02/39986; and in Reslow et al “Drug Delivery Systems & Sciences 2002, Vol 2 No. 4 pp 103-109 describe protein-containing microparticles.

Micronisation is a potentially attractive technique because of its relative simplicity. It involves introducing the material to be formulated into a chamber on a current of turbulent air. It does not require making a solution of macromolecules and so the chemical lability of macromolecules is not an issue. However, the high energy generated in micronisation apparatus would be sufficient to degrade most macromolecules and as such the skilled person would dismiss this approach as impracticable.

The prior art with regard to blending fine particulates with coarser carrier materials to form inhalation formulations is uninformative. It merely provides that the carrier must be inert, and sufficiently coarse to not be inhaled into the deep lung. Suitable carrier materials used in inhalable formulations are mono- or di-saccharides such as lactose, glucose, sucrose or trehalose, sugar alcohols such as mannitol or xylitol, polylactic acid and cyclodextrin.

Applicant has now found that in selecting certain qualities of carrier material, it is possible to blend fine particles containing macromolecules with carrier materials to form inhalable formulations having all the advantages discussed above. Still further, applicant has also now found that it is possible to effect particle-size reduction of macromolecules in a cost-effective manner without degrading or substantially degrading the macromolecules by a micronisation technique wherein carrier material is co-micronised with the macromolecule.

Accordingly, in a first aspect the invention provides an inhalation formulation comprising fine particles containing macromolecules and a carrier material, wherein the carrier material is in crystalline form.

Crystalline carrier materials have excellent bulk properties such as flow properties, and permit the formulation of even sticky or cohesive macromolecules. In this regard, bulk macromolecules provided in lyophilized form tend to be particularly cohesive materials, and the present invention is particularly suited to the formulation of such materials.

Further, unlike amorphous materials, crystalline carrier materials are irregularly shaped, and it is believed, although applicant does not intend to be bound by theory, that fine particles made from macromolecules adhere to the crystalline carrier materials, and are rendered relatively immobile, and therefore less reactive at the molecular level. Amorphous carriers are rather smooth and by contrast, the macromolecules associated with such carriers are relatively mobile and more reactive at the molecular level. Accordingly, labile macromolecules formulated with amorphous carriers are more sensitive when exposed to ambient conditions such as humidity and oxygen.

Preferred crystalline carrier materials are selected from any of those inert carrier materials approved for use in inhalable compositions. Examples of suitable crystalline carrier materials are selected from the group consisting of fructose, saccharose, sucrose, maltose, mannitol, lactose monohydrate, glucose mono-hydrate, xylitol, xylose and sorbitol.

More preferred crystalline carrier materials are those materials displaying adsorption isotherms wherein no or substantially no water is taken up by the carrier at 80% or lower humidity. Most preferred crystalline carrier materials are lactose mono-hydrate or glucose mono-hydrate.

Crystalline carrier materials for use in the present invention are substantially entirely crystalline in form, preferably greater than 95% crystalline, more particularly 99% or greater, although it is not possible to discount that there may be small domains of amorphous material.

Amorphous domains tend to attract ambient moisture and will tend to recrystallise if the ambient moisture is sufficiently high. If there are substantial domains of amorphous material, then recrystallisation can be deleterious to the bulk properties of an inhalation formulation, e.g. the formulation will clump or form a crust. This, in turn, may affect a formulation's ability to be expressed from a dry powder inhaler device with reliable dose uniformity. Accordingly, formulations according to the present invention may employ a ternary ingredient that is provided to sequester any residual moisture. A preferred ternary ingredient in this respect is magnesium stearate.

The use of magnesium stearate may improve the quality of the inhalation formulation and result in improved storage stability of the macromolecule and a reduction of the influence of penetrating moisture on the inhalation formulation, even if dosage forms are stored in conditions of high humidity.

Magnesium stearate may be used in a formulation in a pulverized form. The particle size is not significant, although for ease of blending it is preferably of similar particle size to the coarse carrier material employed.

The fine particles containing macromolecule preferably have a mean mass diameter of between about 1 to about 10 microns. Particles having these dimensions are sufficiently fine to pass through the deep lung and be delivered systemically.

The carrier particles on the other hand should have a mean mass diameter such that they are not inhalable, that is, penetration into the deep lung is foreclosed because of the relatively large particle size. Typically, carrier materials may have mean mass diameter of about 10 microns to 1000 microns, more particularly 20 to 500 microns. Consistent with this range, the skilled person will appreciate that a small proportion of much finer particles can be present in the coarse particles without affecting the function of the carrier material.

Inhalable formulations according to the present invention may contain carrier material in amounts of about 1 to 99% or more by weight, in particular 10 to 99% by weight, more particularly 50 to 99% by weight.

The amount of macromolecule employed in the inhalation formulation will depend upon the nature of the macromolecule, the type and severity of the condition to be treated, and the nature of the subject to be treated. Inhalable formulations according to the present invention may contain 0.1 to 20% by weight macromolecule, in particular 0.1 to 5 weight %, more particularly 0.1 to 2% by weight.

When magnesium stearate is employed as a ternary ingredient, it may be employed in amounts of 0.001 to 10% by weight, more particularly 0.01 to 5% by weight.

The macromolecules for use in the present invention may be provided in their bulk form, for example as lyophilized masses or in crystalline form. Alternatively, the macromolecules may be provided pre-formulated. For example they may be provided in conjugated form, e.g. conjugated with polytethylene glycols in a process that has become known as ‘pegylation’. Such conjugated proteins are disclosed in U.S. Pat. No. 6,136,563 which is incorporated herein by reference. Alternatively, they may be employed in microparticles as more fully described in U.S. Pat. No. 6,051,256 and in U.S. Pat. No. 5,981,719 both of which references are incorporated herein by reference. In yet another alternative, the macromolecules may be incorporated into Biosphere® microparticles as more fully described in U.S. Pat. No. 4,822,535; U.S. Pat. No. 6,120,787; WO02/28908; WO 02/39986; and in Reslow et al “Drug Delivery Systems & Sciences 2002, Vol 2 No. 4 pp 103-109, all of which documents are incorporated herein by reference.

The aforementioned microparticles may be formed with mean mass diameters suitable for inhalation, e.g. from about 1 to 10 microns, and as such no further particle size reduction would be necessary before blending with carrier material.

In contrast, when provided in bulk form, the bulk macromolecule will usually have to undergo particle-size reduction before it can be blended with the carrier material. Particle-size reduction methods form another aspect of the present invention. A method comprises the step of co-micronising a macromolecule-containing material with a carrier material.

Whereas micronisation processes subject materials to very high mechanical and even thermal stresses, which ordinarily may contain sufficient energy that would destroy the tertiary structure of a macromolecule, it is believed that the use of a carrier material in a co-micronisation process diverts energy away from the macromolecule. It is believed, although the applicant is not intending to be bound by any theory, that the presence of the carrier material essentially provides an energy sink, diverting excess energy away from the macromolecule. As a result, macromolecule retains all or substantially all of its biological activity despite being subjected to a micronisation process. In this manner, particle-size reduction can be achieved according to a simple and cost-effective method.

The present invention also provides in yet another of its aspects a fine particle material comprising a co-micronised mixture of a macromolecule and a carrier material. The fine particulate material is inhalable and preferably has a mean mass diameter of about 1 to 10 microns as discussed above.

The co-micronised mixture may contain from 0.1 to 80% by weight of macromolecule, the remainder of the mixture being provided by the carrier material. However, optionally one can add a moisture sequestering agent such as magnesium stearate to the mixture, particularly if this mixture is to be stored for an extended period of time before blending with a carrier material to form inhalable formulations. Substantially all of the magnesium stearate that would be employed in the inhalable formulation can be added to the co-micronised mixture, or only a portion of it may be added at the discretion of the formulator. The magnesium stearate may be added to the other ingredients before micronisation, or it may be added to the co-micronised mixture after the micronisation step.

The carrier materials used in the co-micronised mixtures are preferably provided in crystalline form for the reasons discussed above. However, having regard to the amount of carrier material employed in the co-micronised mixture, one might use an amorphous form. This is particularly the case if the amount of carrier material employed is small, and/or if magnesium stearate is employed as a ternary ingredient in the co-micronised mixture.

Macromolecule used in the present invention include all manner of proteins, peptides, oligopeptides, polypeptides, polyamino acids nucleic acid, polynucleotides, oligo-nucleotides and high molecular weight polysaccharides.

Examples of macromolecules that find use in the present invention are:—

Albumins (preferably, human serum Insulin; albumin); BSA; IgG; IgM; insulin; GCSF; GMCSF; LHRH; VEGF; hGH; lysozyme; alpha-lactoglobulin; basic fibroblast growth factor basic fibroblast growth factor; (bFGF); asparaginase; tPA; urokin-VEGF; chymotrypsin; trypsin; streptokinase; interferon; carbonic anhydrase; ovalbumin; glucagon; ACTH; oxytocin; phosphorylase b; alkaline phos-secretin; vasopressin; levothyroxin; phatase; beta-galactosidase; parathyroid hormone, calcitonin; fibrinogen; polyaminoacids (e.g., DNAse, alpha1 antitrypsin; polylysine, polyarginine); angiogenesis inhibitors or pro-immunoglobulins (e.g., antibodies); moters; somatostatin and analogs; casein; collagen; gelatin; soy protein; and cytokines (e.g., interferon, interleukin); immunoglobulins;
Physiologically active proteins such as peptide hormones, cytokines, growth factors, factors acting on the cardiovascular system, factors acting on the central and peripheral nervous systems, factors acting on humoral electrolytes and hemal substances, factors acting on bone and skeleton, factors acting on the gastrointestinal system, factors acting on the immune system, factors acting on the respiratory system, factors acting on the genital organs, and enzymes;
Hormones and hormone modulators including insulin, proinsulin, C-peptide of insulin, a mixture of insulin and C-peptide of insulin, hybrid insulin cocrystals (Nature Biotechnology, 20, 800-804, 2002), growth hormone, parathyroid hormone, luteinizing hormone-releasing hormone (LH-RH), adrenocorticotropic hormone (ACTH), amylin, oxytocin, luteinizing hormone, (D-Tryp6)-LHRH, nafarelin acetate, leuprolide acetate, follicle stimulating hormone, glucagon, prostaglandins, estradiols, testosterone, and other factors acting on the genital organs and their derivatives, analogues and congeners. As analogues of said LH-RH, such known substances as those described in U.S. Pat. Nos. 4,008,209, 4,086,219, 4,124,577, 4,317,815 and 5,110,904 can be mentioned;
Hematopoietic or thrombopoietic factors include, among others, erythropoietin, granulocyte colony stimulating factor (G-CSF), granulocyte-macrophage stimulating factor (GM-CSF) and macrophage colony stimulating factor (M-CSF), leukocyte proliferation factor preparation (Leucoprol, Morinaga Milk), thrombopoietin, platelet proliferation stimulating factor, megakaryocyte proliferation (stimulating) factor, and factor VIII;
Therapeutic factors acting on bone and skeleton and agents for treating osteoporosis including bone GLa peptide, parathyroid hormone and its active fragments (osteostatin, Endocrinology 129, 324, 1991), histone H4-related bone formation and proliferation peptide (OGP, The EMBO Journal 11, 1867, 1992) and their muteins, derivatives and analogs thereof;
Enzymes and enzyme cofactors including pancrease, L-asparaginase, hyaluronidase, chymotrypsin, trypsin, tPA, streptokinase, urokinase, pancreatin, collagenase, trypsinogen, chymotrypsinogen, plasminogen, streptokinase, adenyl cyclase, and superoxide dismutase (SOD);
Vaccines include Hepatitis B, MMR (measles, mumps, and rubella), and Polio vaccines;
Growth factors include nerve growth factors (NGF, NGF-2/NT-3), epidermal growth factor (EGF), fibroblast growth factor (FGF), insulin-like growth factor (IGF), transforming growth factor (TGF), platelet-derived cell growth factor (PDGF), and hepatocyte growth factor (HGF);
Factors acting on the cardiovascular system including factors which control blood pressure, arteriosclerosis, etc., such as endothelins, endothelin inhibitors, endothelin antagonists described in EP 436189, 457195, 496452 and 528312, JP [Laid Open] No. H-3-94692/1991 and 130299/1991, endothelin producing enzyme inhibitors vasopressin, renin, angiotensin I, angiotensin II, angiotensin III, angiotensin I inhibitor, angiotensin II receptor antagonist, atrial naturiuretic peptide (ANP), and antiarrythmic peptide;
Factors acting on the central and peripheral nervous systems including opioid peptides (e.g. enkephalins, endorphins), neurotropic factor (NTF), calcitonin gene-related peptide (CGRP), thyroid hormone releasing hormone (TRH), salts and derivatives of TRH [JP [Laid Open] No. 50-121273/1975 (U.S. Pat. No. 3,959,247), JP [Laid Open] No. 52-116465/1977 (U.S. Pat. No. 4,100,152)], and neurotensin;
Factors acting on the gastrointestinal system including secretin and gastrin;
Factors acting on humoral electrolytes and hemal substances including factors which control hemagglutination, plasma cholesterol level or metal ion concentrations, such as calcitonin, apoprotein E and hirudin. Laminin and intercellular adhesion molecule 1 (ICAM 1) represent exemplary cell adhesion factors;
Factors acting on the kidney and urinary tract including substances which regulate the function of the kidney, such as brain-derived natriuretic peptide (BNP), and urotensin;
Factors which act on the sense organs including factors which control the sensitivity of the various organs, such as substance P;
Chemotherapeutic agents, such as paclitaxel, mytomycin C, BCNU, and doxorubicin;
Factors acting on the immune system including factors which control inflammation and malignant neoplasms and factors which attack infective microorganisms, such as chemotactic peptides and bradykinins; and
Naturally occurring, chemically synthesized or recombinant peptides or proteins which may act as antigens, such as cedar pollen and ragweed pollen, and these materials alone or together with coupled to haptens, or together with an adjuvant.

In order to facilitate the preparation inhalation formulations as describe herein above there is provided, in a further aspect of the present invention, a process for the preparation of inhalation formulations as described above comprising a step of blending fine particles containing a macromolecule with a crystalline carrier material.

Blending may be carried out in a manner known per se using known apparatus. However, considering the beneficial properties of the crystalline carrier materials, and the relative ease of working with which sticky or cohesive and therefore recalcitrant macromolecule-containing materials, the blending process can be carried out using low-shear equipment such as a tumble mixer. In this manner, one is able to obtain inhalation formulations that comprise uniformly mixed fine particles and carrier material, without resorting to relatively harsh high shear conditions as are typical in blending processes using, for example, ball milling techniques.

When employed, a ternary excipient such as magnesium stearate may be added to the blend. The exact order of mixing ingredients is not important, and for convenience carrier, macromolecule and magnesium stearate are mixed together and blended.

In one embodiment of the process invention, the fine particles containing macromolecule are pre-formed as microparticles containing macromolecule.

In an alternative embodiment, the fine particles are formed by co-micronising macromolecule and a carrier material to produce a co-micronised mixture having the requisite mean mass diameter referred to herein above.

The co-micronised mixture may be prepared in a suitable micronisation apparatus such as a Jet Mill by blending the macromolecule and carrier material and feeding the blend into a micronisation chamber whereupon the blend is reduced to fine particles by the shearing action of high velocity compressed air streams in a manner known per se.

A process of making formulations of the present invention are more fully described in the Example set forth below.

Inhalation formulations of the present invention may be filled in suitable containers, and sealed according to techniques well known in the art. The packages thus formed represent another aspect of the present invention. The packages are adapted to fit into and cooperate with Dry Powder Inhaler (DPI) devices in order to permit delivery of the inhalation formulation to a patient. Packages are well known in the art and are adapted to receive inhalation formulations consisting of single, tens or even hundreds of therapeutic doses. The term “therapeutic dose(s)” as used herein means an amount of inhalation formulation containing a requisite amount of macromolecule to illicit a therapeutic effect, e.g. to alleviate, prevent or inhibit the particular condition to be treated, when delivered to a patient. There is no typical therapeutic dose; it depends largely on the nature of the macromolecule, the condition of the patient, and the nature and severity of the condition to be treated. A therapeutic dose may range between as little as 1 ng/kg to as much as 10 mg/kg, more particularly 20 ng/kg to 1 mg/kg.

In another aspect of the invention there is provided a DPI device containing an inhalation formulation as herein above described.

Inhalation formulations obtained according to the present invention can be employed in all manner of dry powder inhaler devices commonly available in the art. They are particularly suitable for use in multidose DPI devices, which contain a powder reservoir. Particularly useful DPI devices are described in WO 97/20589 which is hereby incorporated by reference.

A therapeutic dose may be delivered with one or more actuations of the DPI device. This is because the amount of powder that can be delivered to a patient without irritating the patient, e.g. making the patient cough, is limited to about 50 mg per actuation, more particularly 25 mg per actuation. Accordingly, depending on the nature of the macromolecule and the nature and severity of the condition to be treated, one or more actuations may be necessary per number of hours, per day, for any number of days, weeks, months and so-forth.

Inhalation formulations as described above in relation to the present invention are possessed of many advantages. The use of crystalline carrier material enables labile macromolecules to be blended and formed into inhalation formulations without, or substantially without loss of the biological activity of macromolecules. The inhalation formulations are provided in free-flowing form consistent with the crystalline nature of the carrier material particles.

Because the carrier material is crystalline or substantially crystalline, it is able to be blended easily with sticky or cohesive macromolecule materials. Furthermore blending is neither protracted nor does it employ harsh conditions, and therefore labile materials can be blended without loss, or substantially without loss, of their biological activity. Still further, the formulation is not prone to clumping or forming a crust. Because the integrity of the inhalation formulation as a free-flowing powder is maintained, therapeutic dosage forms can be expressed from a DPI device with excellent dosage uniformity even after prolonged periods of storage under conditions of high humidity.

In addition to excellent dosage uniformity, the proportion of the dosage form that contains fine particles that can penetrate deep into the lung and be delivered systemically is very high.

As is well known, a dosage expressed from a DPI device contains coarse particles and fine particles. It is the fine particles that are able to enter the deep lung and be delivered systemically. Whereas a package may be filled with very fine particles containing macromolecules, during storage the powder quality can change for the reasons set forth above, such that the fine macromolecule particles cannot be expressed with high efficiency from the device. The proportion of a delivered dose that is in such fine particulate form is commonly expressed in terms of its Fine Particle Fraction or FPF.

FPF is expressed as the ratio of the fine particle content to the total content of the dosage expressed from a DPI device. FPF is measurable by determining the aerodynamic particle size distribution of the expressed inhalation formulation. It can be measured using Compendial apparatus and methods such as the Andersen Cascade Impactor or the Multi-stage Liquid Impinger described in pharmacopoeial test monographs such as are described in US Pharmacopoeia and European Pharmacopoeia.

It is a characteristic of the present invention that the inhalation formulations are able to express a dosage that contains macromolecules with greater than 50% FPF, more particularly greater than 70%, still more particularly greater than 90%, e.g. greater than 95%.

Medicaments containing macromolecules with very high Fine Particle Fraction are naturally very beneficial for physician and patient alike. The physician is able to provide a greater systemic effect for a given dose of medicament, or alternatively, the physician can administer lower doses to a patient (and therefore lower volumes) for a given systemic effect.

The invention will now be further illustrated with reference to the following examples.

### EXAMPLE 1

10 g fine lactose (Pharmatose 325 M, DMV International/Netherlands) and 0.1 g of magnesium stearate are sieved through a 250 μm mesh and blended in a in a tumble blender at 32 rpm for 20 minutes to yield a pre-blend of lactose and magnesium stearate. 0.2 g of lyophilized bulk protein having a molecular weight of about 13 KDa and 1.8 g of the magnesium stearate-lactose pre-blend are weighed and both sieved through a 250 μm mesh into a suitable blending vessel (container). The container content is blended in a tumble blender at 32 rpm for 20 minutes. The resulting blend is micronized in an air-jet mill (Hosokawa Alpine 50AS) at an inlet air pressure of 8 bar to yield 1.6 g of micronized blend. The micronized blend and 5.4 g of lactose pre-blend are then sieved together through a 250 μm mesh into a blending vessel of appropriate size. The container content is blended at 32 rpm for 10 minutes in a Tumble Blender. The resulting dry powder formulation is filled into a dry powder inhaler device (SkyeHaler®). The fine particle fraction of the delivered dry powder formulation containing 2.3% w/w of protein is more than 50% of intact protein based on the total recovered dose when tested in-vitro with the Andersen Cascade Impactor at 60 L/min according to Compendia, methodology.

After 6 months storage of the finished drug product at 40° C./75% R.H. the fine particle fraction remains at more than 50%.

1. A powder formulation for inhalation comprising a macromolecule and a crystalline carrier material.

2. The formulation according to claim 1 wherein the carrier material is selected from the group consisting of fructose, saccharose, sucrose, maltose, mannitol, lactose monohydrate, glucose mono-hydrate, xylitol, xylose and sorbitol.

3. The formulation of claim 1, further comprising magnesium stearate.

4. The formulation of claim 1, wherein the macromolecule is selected from the group consisting of proteins, peptides, oligopeptides, polypeptides, polyamino acids nucleic acid, polynucleotides, oligo-nucleotides and high molecular weight polysaccharides.

5. (canceled)

6. The formulation of claim 1, wherein the macromolecule is provided in bulk form, in pegylated form, or is provided in the form of microparticles.

7. The formulation of claim 1, wherein the macromolecule is provided in micronised form.

8. A method of forming the formulation of claim 1, comprising the step of blending a fine particles containing a macromolecule with a carrier material.

9. The method of claim 8, wherein the fine particles of macromolecule are formed in a process whereby bulk macromolecule is co-micronised with a carrier material.

10. A fine particulate material containing co-micronised macromolecule and carrier material.

11. A package comprising single or multiple therapeutic doses of the inhalation formulation of claim 1.

12. A dry powder inhaler equipped with a package as defined in claim 11.

13. A method of administering a macromolecule to the circulatory blood flow of a patient comprising the step of administering to the patient a therapeutic dose of the inhalation formulation of claim 1.
